(19) United States
(12) Patent Application Publication
(10) Pub. No.: US 2018/0035922 A1
(43) Pub. Date: Feb. 8, 2018

(54) METHOD FOR BIOMETRIC HUMAN IDENTIFICATION BASED ON ELECTROCARDIOGRAM AND PTT SMART WATCH SYSTEM USING THE SAME METHOD

(71) Applicant: Dodotdo Co., Ltd, Seoul (KR)

(72) Inventors: In Gyeom KIM, San Diego, CA (US); So Yeong SIM, Seoul (KR)

(21) Appl. No.: 15/286,587

(22) Filed: Oct. 6, 2016

(30) Foreign Application Priority Data
Aug. 2, 2016 (KR) 10-2016-0098562

(51) Int. Cl. A61B 5/117; H04W 68/00; G01S 19/13; G08B 3/10; A61B 5/0402; A61B 5/00; H04W 4/10; G08B 6/00

(52) U.S. Cl. CPC A61B 5/117; H04W 4/10; H04W 68/005; G08B 6/00; G08B 3/1033; A61B 5/0402; A61B 5/0006; A61B 5/0022; A61B 5/681; G01S 19/13; H04B 2001/3861

(57) ABSTRACT

A PTT smart watch system using a biometric human identification (HID) method based on electrocardiogram which provides a PTT service through interworking with a server including an electrocardiogram database, the PTT smart watch system comprising: a plurality of user terminals enabling biometric HID and PTT communication; and a server configured to receive electrocardiogram data and a PTT message from a first user terminal of the plurality of user terminals, transmit the electrocardiogram data to an electrocardiogram database, and transmit the PTT message to at least one second user terminal of the plurality of user terminals which enters a corresponding PTT channel when biometric HID is identified from the electrocardiogram database. A biometric HID method based on electrocardiogram enhances security in a PTT communication system, and allows a convenient use. In addition, through interworking with a health care application in a smartphone using electrocardiogram data, a user's health may be managed.

### TECHNICAL FIELD

Exemplary embodiments relate to a push-to-talk (PTT) mobile communication device, and more particularly, to a biometric human identification method based on electrocardiogram and a smart watch system using the same method.

### BACKGROUND ART

In recent times, smart watches integrated with mobile phone functions are garnering attention. As mobile phones started adopting not only telephone networks but also Internet networks through which personal information, such as documents, finances and the like, are transmitted and received, the awareness on issues relating to mobile phone security is becoming prevalent.

The more complicated a user identification process becomes, the more inconvenient mobile phone users feel. To address such issue, a biometric identification scheme may be introduced to provide mobile phone security and user identification in a simple and convenient manner.

Biometrics refers to metrics related to personal information using physiological characteristics and behavioral characteristics as identifiers. Examples include, but are not limited to fingerprint, retina, iris recognition, face recognition, hand, palm veins, voice, signature or DNA.

Fingerprint recognition is a technique of identifying a certain flow in a sweat gland protuberant in a finger, exhibiting efficiency in reliability and stability in performance. Such fingerprint recognition employs thinning, Fourier transform, wavelet transform, neural networks, or Fuzzy logic, and requires a process of de-noising and correction. Also used for fingerprint recognition are syntactic, statistical or rule-based schemes, neural networks, chain codes, or ridge-following.

However, fingerprint recognition has disadvantages in that geometrical deformation may occur in fingerprints recognized based on the resolution of a fingerprint image, pixel distribution characteristics, a scanning scheme of a fingerprint input sensor and that ridge number extraction and the use of fingerprint recognition may be sensitive to the rotation and deformation of fingerprints and has low reproducibility.

Iris recognition refers to a technique of identifying individuals based on the form of an iris tissue. Although iris recognition has relatively low recognition error rate, it requires precise focal distance between an iris of a user and a scanner and has difficulty obtaining precise iris pattern when a user moves. When capturing an image of an iris, the color of an iris image may change, and thus, iris recognition may be made difficult in case of wearing a contact lens, such as a color lens.

Retina scanning recognizes the pattern of blood vessels within human retina. In retina scanning, the pattern of blood vessels may change depending on the presence of glasses or a contact lens, or blood alcohol concentration. Since it requires light irradiation from a scanner to eye during retina scanning, there is a risk of eye contamination due to direct contact of eye and the scanner.

Face recognition identifies individuals by capturing an image of a face in a non-contact manner, extracting a face part from the captured image, and analyzing points for each characteristic of the face. Despite its simple process, it still faces challenges in commercialization thereof, because the shape of face may vary depending on an angle of face, facial expressions, or age and a matching rate is low.

Palm vein pattern recognition identifies the pattern of palm veins using a contact-type infrared capturing device. Although it has an advantage in that vein patterns are non-duplicable, it is difficult to establish a database of vein patterns and requires rather expensive device.

In voice recognition, there may be various obstructions, such as change in human voice in course of time or noise, and voice recognition may have risks of voice fabrication/falsification.

### DISCLOSURE OF INVENTION

### Problem to be Solved by the Invention

The present invention aims to enhance security using electrocardiogram biometrics, and exemplary embodiments of the present invention provide a biometric human identification method based on electrocardiogram and a PTT communication system using the same method.

However, the present invention should not be construed as being limited to the exemplary embodiments set forth herein, and rather, these exemplary embodiments are provided so that this disclosure will fully convey the scope of the invention to those skilled in the art.

### Means for Solving Problem

According to an exemplary embodiment of the present invention, a PTT smart watch system using a biometric human identification (HID) method based on electrocardiogram which provides a PTT service through interworking with a server including an electrocardiogram database, the PTT smart watch system may include: a plurality of user terminals enabling biometric HID and PTT communication; and a server configured to receive electrocardiogram data and a PTT message from a first user terminal of the plurality of user terminals, transmit the electrocardiogram data to an electrocardiogram database, and transmit the PTT message to at least one second user terminal of the plurality of user terminals which enters a corresponding PTT channel when biometric HID is identified from the electrocardiogram database.

The server may be provided in a hospital to store the electrocardiogram database transmitted from the first user terminal, and wherein a user having the first user terminal performs PTT communication with a doctor having the second user terminal.

According to another exemplary embodiment of the present invention, a PTT smart watch using a biometric human identification (HID) method based on electrocardiogram which provides a PTT service through interworking with a server including an electrocardiogram database, the PTT smart watch may include: a communication unit configured to modulate a frame applied to PTT communication to a PTT frequency signal to thereby transmit the modulated frame via an antenna, and demodulate the frequency signal received from the antenna to the frame; an input unit configured as a user input interface comprising a plurality of input keys, the input keys comprising a PTT button configured to support a PTT function in PTT communication; a biometric unit configured to perform a biometric HID method based on electrocardiogram; an output unit configured to output voice data by converting voice data input from a microphone into the frame to transmit the frame to the communication unit in a PTT transmission mode, and extracting voice data from the frame demodulated in the communication unit to output the voice data through a speaker in a PTT reception mode; a memory unit configured to store biometric HID data and electrocardiogram data, the electrocardiogram data storing and inputting or outputting an electrocardiogram identification program performed in a control unit is stored and input or output; an interface unit configured to mutually transmit signals in respective units; and the control unit configured to control the communication unit, input unit, and the output unit so as to execute a PTT communication-enabled mode for performing PTT communication when the PTT button is pushed and execute a PTT reception mode when the PTT button is released.

The communication unit may include at least one of: a broadcast reception module configured to receive a broadcast signal and broadcast-related information from an external broadcast management server through a broadcast channel; a mobile communication module configured to transmit/receive wireless signals with a base station, a repeater, a femtocell, or a radio remote head (RRH) through a wireless interface of a mobile communication network; a wireless Internet communication module embedded in a wearable device or externally provided through a predetermined interface terminal; a local communication module for local communication; and a global positioning system (GPS) reception module configured to verify or obtain location information of the PTT smart watch system using a signal received through satellite.

The output unit may include at least one of: a display module configured to display information processed in the PTT smart watch system; a sound output module configured to output a predetermined sound alarm signal to inform a user when a voice message is received from another user through a PTT application from the wireless communication unit; an alarm module configured to output a signal for notifying an occurrence of an event in the PTT smart watch system; and a vibration module configured to generate various haptic effects for the user to sense.

According to another exemplary embodiment of the present invention, a biometric human identification (HID) method based on electrocardiogram which is provided with a biometric HID device based on electrocardiogram and interworks with a PTT server to provide a PTT service, the method may include: receiving an input of an electrocardiogram waveform of a user and removing the baseline noise of the electrocardiogram waveform; determining a base form for each waveform to determine a base form of the entire waveform for each user; evaluating a change in the base form for each waveform to classify a corresponding waveform; extracting characteristics of the corresponding waveform based on the extracted waveform so as to enable user identification; generating identification information which allows user identification using a code set based on the extracted characteristics to thereby perform user identification; performing a PTT communication-enabled mode when a user is identified; and performing PTT transmission and PTT reception.

The determining of a base form for each waveform to determine a base form of an entire waveform for each user may further include identifying pulse forms grouped in a simple manner such as low below (LB), medium below (MB), high below (HB), low up (LU), medium up (MU), or high up (HU).

The generating of identification information which allows user identification using a code set based on the extracted characteristics to thereby perform user identification may further include aligning a sequence of HID for each individual through an HID database server such that precision for HID and identification rate increase as a greater amount of heartbeat rate electrocardiogram data is collected.

The generating of identification information which allows user identification using a code set based on the extracted characteristics to thereby perform user identification may further include performing a login into an account to perform HID.

### Effect of the Invention

According one or more exemplary embodiments of the present invention, a biometric human identification method based on electrocardiogram enhances the security in a PTT communication system in a simple and convenient manner.

The method may interwork with a health care application in a smartphone to thereby manage user's health based on electrocardiogram data.

### BRIEF DESCRIPTION OF DRAWINGS

FIG. 1 is a schematic diagram illustrating a smart watch PPT system using a biometric human identification method based on electrocardiogram according to an exemplary embodiment.

FIG. 2 is a diagram illustrating a smart watch PPT device according to an exemplary embodiment.

FIG. 3 is a flowchart illustrating a smart watch PPT method according to an exemplary embodiment.

FIG. 4 is a diagram illustrating a waveform of an electrocardiogram identification (HID) of an individual according to an exemplary embodiment.

FIG. 5 is a diagram illustrating a user identification operation based on electrocardiogram.

### EXPLANATIONS OF LETTERS OR NUMERALS

100a, 100b: User terminals

110: Communication unit

111: Broadcast reception module

112: Mobile communication module

113: Router

114: RF

115: TRS

116: Wireless Internet communication module

117: Local communication module

118: GPS reception module

120: Input unit

130: Biometric identification unit

140: Control unit

150: Output unit

151: Display module

153: Sound output module

155: Alarm module

157: Vibration module

160: Memory unit

170: Interface unit

### BEST MODE(S) FOR CARRYING OUT THE INVENTION

Advantages and features of the invention and methods for achieving them will be made clear from exemplary embodiments described below in detail with reference to the accompanying drawings. The invention may, however, be embodied in many different forms and should not be construed as being limited to the exemplary embodiments set forth herein. Rather, these exemplary embodiments are provided so that this disclosure will be thorough and complete, and will fully convey the scope of the invention to those skilled in the art. The invention is merely defined by the scope of the claims. Therefore, well-known constituent elements, operations and techniques are not described in detail in the exemplary embodiments in order to prevent the invention from being obscurely interpreted. Like reference numerals refer to like elements throughout the specification. The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items.

The terminology for a module used herein may be interpreted as including software, hardware or a combination thereof. For example, software may include machine language, firmware, embedded code, and application software. Examples of hardware may include a circuit, a processor, a computer, a direct circuit, a direct circuit core, a sensor, a micro-electro-mechanical system (MEMS), a manual device, or a combination thereof.

Exemplary embodiments will now be described more fully hereinafter with reference to the accompanying drawings. Hereinafter, a PTT smart watch system using a biometric human identification (HID) method based on electrocardiogram will be referred to as a “PTT smart watch system.”

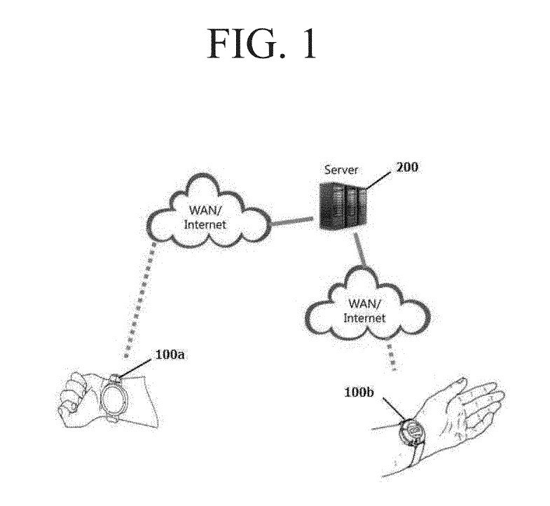

FIG. 1 is a schematic diagram illustrating a smart watch PPT system using a biometric HID method based on electrocardiogram according to an exemplary embodiment.

Referring to FIG. 1, a biometric identification smart watch system provides a PTT service through interworking with a server including an electrocardiogram database. The biometric identification smart watch system comprises: a plurality of user terminals (100a, 100b) enabling biometric identification and PTT communication; and a server (200) configured to receive electrocardiogram data and a PTT message from a first user terminal (100a) of the plurality of user terminals, transmit the electrocardiogram data to an electrocardiogram database, and transmit the PTT message to at least one second user terminal (100b) of the plurality of user terminals which enters a corresponding PTT channel when biometric HID is identified from the electrocardiogram database.

The first user terminal (100a) measures an electrocardiogram of a user, compares a waveform of the electrocardiogram of the user with an existing electrocardiogram waveform, identifies the user, and transmits data on the electrocardiogram of the user to the server (200). The server (200) stores data on the electrocardiogram of the user and transmits a signal to enable PTT communication with the second user terminal (100b).

The user of the second user terminal (100b) receives a signal of the first user terminal (100a) transmitted from the server (200) and performs PTT communication.

In an exemplary embodiment, the user terminals (100a, 100b) are provided for efficient mobile communication of a wearable smart watch. However, such small-sized wearable smart watches require an identification technique for more rapid PTT communication while ensuring security. Since screens for wearable smart watches are small, a user identification technique for wearable smart watches is significant.

In an exemplary embodiment, the user terminals (100a, 100b) transmit/receive information with the server (200) via the wireless access network (WAN) or the Internet, and the user terminals (100a, 100b) transmit/receive information with each other via a mutual mobile communication network.

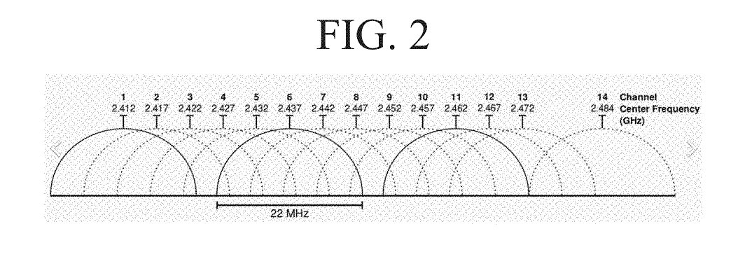

Referring to FIG. 2, a PTT smart watch system in 802.11b, 802.11g, and 802.11n uses a spectrum in a range from 2.400 GHz to 2500 GHz and an industry-science-medical (ISM) band 802.11a and 802.11n uses a band in a range from 4.915 to 5.825 GHz, which is a higher frequency range.

Although 2.4 GHz and 5 GHz bands are commonly used, the respective spectra are divided into channels having a center frequency and a bandwidth, and into radio and TV broadcast bands in an analog scheme. A 2.4 GHz band, which is divided into 14 channels with an interval of 5 MHz, starts from channel 1 and has a center frequency at 2.412 GHz. Communication in such bandwidths is used to increase mobility of a PTT smart watch and enable low power consumption.

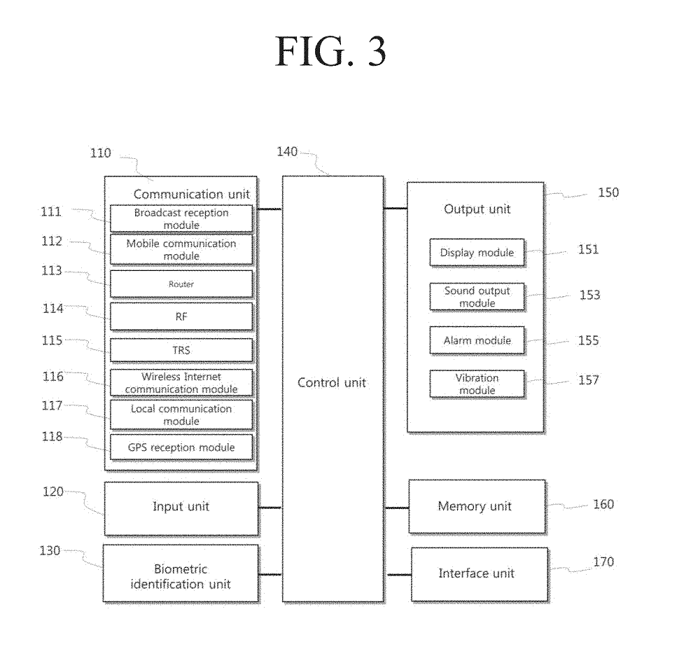

FIG. 3 is a flowchart illustrating a smart watch PPT method according to an exemplary embodiment.

Referring to FIG. 3, PTT user terminals (100a, 100b) using a biometric HID method based on electrocardiogram which provides a PTT service through interworking with a server (200) including an electrocardiogram database may include: a communication unit (110) configured to modulate a frame applied to PTT communication to a PTT frequency signal to thereby transmit the modulated frame via an antenna, and demodulate the frequency signal received from the antenna to the frame; an input unit (120) configured as a user input interface comprising a plurality of input keys, the input keys comprising a PTT button configured to support a PTT function in PTT communication; a biometric unit (130) configured to perform a biometric HID method based on electrocardiogram; an output unit (150) configured to output voice data by converting voice data input from a microphone into the frame to transmit the frame to the communication unit (110) in a PTT transmission mode, and extracting voice data from the frame demodulated in the communication unit (110) to output the voice data through a speaker in a PTT reception mode; a memory unit (160) configured to store biometric HID data and electrocardiogram data, the electrocardiogram data storing and inputting or outputting an electrocardiogram identification program performed in a control unit (140) is stored and input or output; an interface unit (170) configured to mutually transmit signals in respective units; and a control unit (140) configured to control the communication unit (110), input unit (120), and the output unit (150) so as to execute a PTT communication-enabled mode for performing PTT communication when the PTT button is pushed and execute a PTT reception mode when the PTT button is released.

In an exemplary embodiment, the PTT user terminals (100a, 100b) may include the communication unit (110), the input unit (120), the biometric unit (130), the control unit (140), the output unit (150), the memory unit (160), the interface unit (170), and a power supply (180) with reference to FIG. 3.

The communication unit (110) may include at least one module that enables wireless communication between the user terminal (100a) and the user terminal (100b). The communication unit (110) may include at least one of a broadcast reception module (111), a mobile communication module (112), a wireless Internet communication module (115), a local communication module (117) and a global positioning system (GPS) reception module (119).

The broadcast reception module (111) may receive a broadcast signal and broadcast-related information from an external broadcast management server through a broadcast channel.

The mobile communication module (112) may include a router (113), a trunk radio system (TRS) (114), an RF (115), and transmit/receive wireless signals with a base station, a repeater, a femtocell, or a radio remote head (RRH) via a wireless interface of mobile communication.

The router (113) is a device for sharing two different frequency signals via a single route. In a case where the RF (114) and the TRS (115) have separate antennas, there is no need for such router (113).

The RF (114) performs wireless communication according to a wireless communication protocol such as wideband code division multiple access (WCDMA). The RF (114) may include an RF transmitter that up-converts and amplifies a frequency of a signal transmitted therefrom and an RF receiver that no-noise amplifies a signal received thereto and down-converts a frequency.

The TRS (115) performs wireless communication according to a communication protocol in a TRS using a trunked radio system network. The TRS uses a very high frequency (VHF) or an ultra high frequency (UHF) on a charged basis, and forms a radio frequency (RF) distance in a range of 10 Km to 20 Km.

The wireless Internet module (116) is a transception module for wireless Internet connection. The wireless Internet module (116) may be embedded in a wearable device (100) or externally provided through a predetermined interface terminal.

A local communication module (117) for local communication is a module for local communication.

A GPS reception module (118) is a module configured to verify or obtain location information of the PTT smart watch system (100) using a signal received through satellite.

The input unit (120) is configured to operate as a number key for inputting number and character data and a function key for setting various functions. Examples of the function key may include speaker ON/OFF buttons, a volume control button, and a PTT button.

The biometric unit (130) conducts biometric HID method based on electrocardiogram. The biometric HID method based on electrocardiogram conducted in the biometric unit (130) may be conducted in the smart watches (100a, 100b) or the server (200). The biometric unit (130) may use electrocardiogram, fingerprint, retina, iris, face, hand, palm veins, voice, signature, or DNA as biometric identifiers.

The control unit (140) is configured to control the communication unit (110), input unit (120), the output unit (130), the output unit (150), the memory unit (160), and the interface unit (170) so as to execute a PTT communication-enabled mode for performing PTT communication when the PTT button is pushed and execute a PTT reception mode when the PTT button is released.

The output unit (150), for example, a liquid crystal display (LCD) device, displays an image signal output from an image-processing unit on a screen and displays user data output from the control unit (140). Visual, auditory or haptic outputs may be generated by a display module (151) or a sound output module (153).

The display module (151) is configured to display data processed in the PTT smart watch system. The sound output module (153) is configured to output a predetermined sound alarm signal for notifying a user when a voice message from another use is received through a PTT application from the wireless communication unit (110). The sound output module (153) may output audio data stored in the memory unit (160)

An alarm module (155) is configured output a signal for notifying an occurrence of an event in the PTT smart watch system.

A vibration module (157) is configured to generate various haptic effects for a user to sense.

The memory unit (160) is configured to store biometric HID data and electrocardiogram data, the electrocardiogram data storing and inputting or outputting an electrocardiogram identification program performed in a control unit (140) is stored and input or output.

The interface unit (170) enables mutual transmission of the respective units.

The power supply unit (180) is configured as a battery for supplying power to the respective functional units. For a maximum battery lifetime, power consumption needs to be significantly reduced. To this end, a mobile phone with a radio communication function may operate in a walkie-talkie mode, which is a family radio communication function, such that power consumption may be significantly reduced through a sleep mode and a wake-up mode.

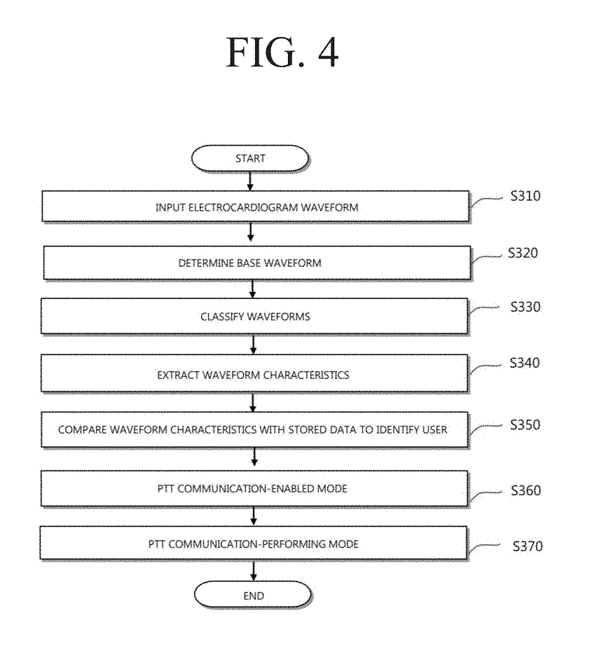

Referring FIG. 4, in another exemplary embodiment, a biometric HI method based on electrocardiogram which is provided with a biometric HID device based on electrocardiogram and interworks with a PTT server to provide a PTT service may include: receiving an input of an electrocardiogram waveform of a user and removing the baseline noise of the electrocardiogram waveform (S310); determining a base form for each waveform to determine a base form of the entire waveform for each user (S320); evaluating a change in the base form for each waveform to classify a corresponding waveform (S330); extracting characteristics of the corresponding waveform based on the extracted waveform so as to enable user identification (S340); generating identification information which allows user identification using a code set based on the extracted characteristics to thereby perform user identification (S350); performing a PTT communication mode when a user is identified (S360); and performing PTT transmission and PTT reception (S370).

In operation S310, the first user terminal (100a) inputs an electrocardiogram waveform of a corresponding user. The first user terminal (100a) removes baseline noise of the electrocardiogram waveform. The removing of baseline noise of the electrocardiogram waveform may be carried out in the server (200).

In operation S320, the first user terminal (100a) determines a base form of each waveform to determine a base form of the entire waveform for each user. The determining of a base form of each waveform to determine a base form of the entire waveform for each user may be carried out in the server (200).

In operation S330, the first user terminal (100a) evaluates a change in the base form for each waveform and classifies a corresponding waveform. The evaluating of a change in the base form for each waveform to classify a corresponding waveform may be carried out in the server (200).

In operation S340, the first user terminal (100a) extracts characteristics of the corresponding waveform based on the extracted waveform so as to enable user identification. The extracting of characteristics of the corresponding waveform based on the extracted waveform so as to enable user identification may be carried out in the server (200).

In operation S350, the first user terminal (100a) generates identification information which allows user identification using a code set based on the extracted characteristics to thereby perform user identification. The generating of identification information which allows user identification using a code set based on the extracted characteristics to thereby perform user identification may be carried out in the server (200).

In operation S360, the first user terminal (100a) performs a PTT communication mode when a user is identified.

In operation S370, when a PTT transmission mode is executed when the PTT button is pushed in a PTT enabled mode of the first user terminal (100a), a signal is transmitted to the second user terminal (100b) to thereby execute a PTT reception mode, and when the PTT button is released from the first user terminal (100a), the PTT reception mode is executed.

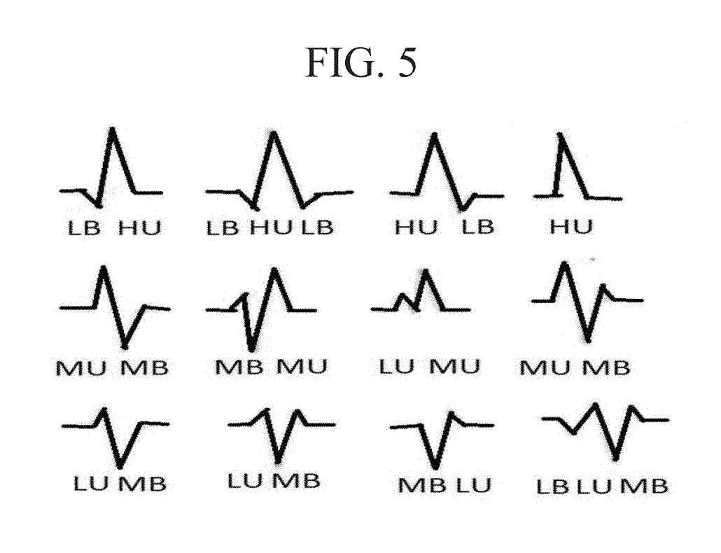

FIG. 5 is a diagram illustrating a user identification operation based on electrocardiogram.

In an exemplary embodiment, a scheme of collecting biometric electrocardiogram data conducts HID by logging-into an account by identifying pulse forms grouped in a simple manner such as low below (LB), medium below (MB), high below (HB), low up (LU), medium up (MU), or high up (HU), and aligns a sequence of HID for each individual through the HID database server (200) such that precision for HID and identification rate increase as a greater amount of heartbeat rate electrocardiogram data is collected.

The sequence of HID for each individual is a series of code connected by a combination of LB, MB, HB, LU, MU, or HU as shown in an exemplary embodiment in FIG. 5. The precision for HID may increase as a greater amount of heartbeat rate electrocardiogram data is collected. Since a main user is rapidly identified and a non-user is not rapidly identified, a greater amount of data does not need to be collected, thus increasing an identification rate. Accordingly, through storing electrocardiogram data in a server and interworking with a health care application of the user terminals (100a, 100b), user's health may be managed.

In an exemplary embodiment, an electrocardiogram data server may be provided in a hospital so that a doctor may use electrocardiogram data of a user to diagnose the user's health, and the user may have a consolation with the doctor as to the user's health in a convenient manner using PTT communication. For example, in an emergency situation, the user may remotely contact the doctor conveniently using PTT communication.

In an exemplary embodiment, the biometric HID method based on electrocardiogram may be implemented in a computer system or recorded in a recording medium. The computer system may include at least one processor, a memory, a user input device, a data communication bus, a user output device and a storage. These elements each perform data communication through the data commutation bus.

The computer system may further include a network interface coupled in a network. The processor may be a central processing unit (CPU) or a semiconductor device that processes a command stored in a memory and/or a storage.

The memory may further include a volatile or non-volatile storing medium in various forms. For example, the memory may include a read only memory (ROM) and a random access memory (RAM).

In an exemplary embodiment, the biometric HID method based on electrocardiogram may be implemented as a computer-readable code in a computer-readable recording medium. The computer-readable recording medium may include all types of recording medium storing data that is interpretable by a computer system.

Examples of the computer-readable recording medium may include a ROM, a RAM, a magnetic tape, a magnetic disk, a flash memory, or an optical data storage. In addition, the computer-readable recording medium may be distributed in a computer system connected by a computer communication network, stored as a code readable in a distribution manner and performed.

From the foregoing, it will be appreciated that various embodiments in accordance with the present disclosure have been described herein for purposes of illustration, and that various modifications may be made without departing from the scope and spirit of the present teachings. Accordingly, the various embodiments disclosed herein are not intended to be limiting of the true scope and spirit of the present teachings. Various features of the above described and other embodiments can be mixed and matched in any manner, to produce further embodiments consistent with the invention.

1. A PTT smart watch system using a biometric human identification (HID) method based on electrocardiogram which provides a PTT service through interworking with a server including an electrocardiogram database, the PTT smart watch system comprising:
a plurality of user terminals enabling biometric HID and PTT communication; and
a server configured to receive electrocardiogram data and a PTT message from a first user terminal of the plurality of user terminals, transmit the electrocardiogram data to an electrocardiogram database, and transmit the PTT message to at least one second user terminal of the plurality of user terminals which enters a corresponding PTT channel when biometric HID is identified from the electrocardiogram database.

2. The PTT smart watch system of claim 1, wherein the server is provided in a hospital to store the electrocardiogram database transmitted from the first user terminal, and wherein a user having the first user terminal performs PTT communication with a doctor having the second user terminal.

3. A PTT smart watch using a biometric human identification (HID) method based on electrocardiogram which provides a PTT service through interworking with a server including an electrocardiogram database, the PTT smart watch comprising:
a communication unit configured to modulate a frame applied to PTT communication to a PTT frequency signal to thereby transmit the modulated frame via an antenna, and demodulate the frequency signal received from the antenna to the frame;
an input unit configured as a user input interface comprising a plurality of input keys, the input keys comprising a PTT button configured to support a PTT function in PTT communication;
a biometric unit configured to perform a biometric HID method based on electrocardiogram;
an output unit configured to output voice data by converting voice data input from a microphone into the frame to transmit the frame to the communication unit in a PTT transmission mode, and extracting voice data from the frame demodulated in the communication unit to output the voice data through a speaker in a PTT reception mode;
a memory unit configured to store biometric HID data and electrocardiogram data, the electrocardiogram data storing and inputting or outputting an electrocardiogram identification program performed in a control unit is stored and input or output;
an interface unit configured to mutually transmit signals in respective units; and
the control unit configured to control the communication unit, input unit, and the output unit so as to execute a PTT communication-enabled mode for performing PTT communication when the PTT button is pushed and execute a PTT reception mode when the PTT button is released.

4. The PTT smart watch of claim 3, wherein the communication unit comprises at least one of:
a broadcast reception module configured to receive a broadcast signal and broadcast-related information from an external broadcast management server through a broadcast channel;
a mobile communication module configured to transmit/receive wireless signals with a base station, a repeater, a femtocell, or a radio remote head (RRH) through a wireless interface of a mobile communication network;
a wireless Internet communication module embedded in a wearable device or externally provided through a predetermined interface terminal;
a local communication module for local communication; and
a global positioning system (GPS) reception module configured to verify or obtain location information of the PTT smart watch system using a signal received through satellite.

5. The PTT smart watch of claim 3, wherein the output unit comprises at least one of:
a display module configured to display information processed in the PTT smart watch system;
a sound output module configured to output a predetermined sound alarm signal to inform a user when a voice message is received from another user through a PTT application from the wireless communication unit;
an alarm module configured to output a signal for notifying an occurrence of an event in the PTT smart watch system; and
a vibration module configured to generate various haptic effects for the user to sense.

6. A biometric human identification (HID) method based on electrocardiogram which is provided with a biometric HID device based on electrocardiogram and interworks with a PTT server to provide a PTT service, the method comprising:
receiving an input of an electrocardiogram waveform of a user and removing the baseline noise of the electrocardiogram waveform;
determining a base form for each waveform to determine a base form of the entire waveform for each user;
evaluating a change in the base form for each waveform to classify a corresponding waveform;
extracting characteristics of the corresponding waveform based on the extracted waveform so as to enable user identification;
generating identification information which allows user identification using a code set based on the extracted characteristics to thereby perform user identification;
performing a PTT communication-enabled mode when a user is identified; and
performing PTT transmission and PTT reception.

7. The method of claim 6, wherein the determining of a base form for each waveform to determine a base form of an entire waveform for each user further comprises identifying pulse forms grouped in a simple manner such as low below (LB), medium below (MB), high below (HB), low up (LU), medium up (MU), or high up (HU).

8. The method of claim 6, wherein the generating of identification information which allows user identification using a code set based on the extracted characteristics to thereby perform user identification further comprises aligning a sequence of HID for each individual through an HID database server such that precision for HID and identification rate increase as a greater amount of heartbeat rate electrocardiogram data is collected.

9. The method of claim 6, wherein the generating of identification information which allows user identification using a code set based on the extracted characteristics to thereby perform user identification further comprises performing a log-in into an account to perform HID.
